(19) United States
(12) Patent Application Publication
(10) Pub. No.: US 2023/0149447 A1
(43) Pub. Date: May 18, 2023

(54) KIT AND METHOD FOR PROVIDING ADDITIONAL DEFENSE AGAINST ALL CORONA VIRUS STRAINS

(71) Applicant: Ahmed Elkoulily, M.D., Lloyd Harbor, NY (US)

(72) Inventor: Ahmed Elkoulily, M.D., Lloyd Harbor, NY (US)

(21) Appl. No.: 17/525,596

(22) Filed: Nov. 12, 2021

(51) Int. Cl. A61K 33/18; A61K 9/00; A61K 31/155; A61K 9/08; A61K 31/045

(52) U.S. Cl. CPC A61K 33/18; A61K 9/0043; A61K 9/0053; A61K 31/155; A61K 9/08; A61K 31/045

(57) ABSTRACT

A kit and method for providing a “second line of defense” against Corona Virus strains. The kit includes at least one nasal swab pre-moistened with an iodine solution, and at least one oral swab pre-moistened with a chlorhexidine solution. The iodine solution can be a solution of betadine 0.5-2.0%, or a povidone solution. The chlorhexidine solution is chlorhexidine 0.09% to 0.12%. The nasal swabs are preferably sealed in a resealable package. The oral swabs are preferably sealed in a separate resealable package. The kit can include alcohol swabs pre-moistened with 60%-70% Isopropyl Alcohol in a disposable sealed package. The method includes using at least one nasal swab pre-moistened with an iodine solution to apply the iodine solution to an inner surface within each of one's nostrils, and using at least one oral swab pre-moistened with chlorhexidine solution to apply the chlorhexidine solution to an inner surface of one's mouth.

### FIELD OF THE INVENTION

This invention relates generally to viral defense, and particularly to topical chemoprotection against viruses.

### BACKGROUND OF THE INVENTION

Corona viruses are a group of related RNA viruses that cause diseases, such as respiratory tract infections, that can range from mild to lethal. Mild illnesses in humans include some cases of the common cold, while more lethal varieties can cause SARS, MERS, and COVID-19.

Methods to slow or stop the spread of COVID-19 infection include: wearing masks, social distancing, mRNA vaccination, and/or various drug therapies. Nevertheless, COVID-19 remains a threat, especially as new variants are discovered that appear resistant to the known methods.

### SUMMARY OF THE INVENTION

The kit of the invention is a ready-to-use kit for protecting two major portals of entry of viruses into the body (the nose and the mouth) using pre-moistened swabs.

Each kit includes at least two pre-moistened nasal swabs and at least one premoistened oral swab. Optionally, pre-moistened alcohol pads are also included for sterilizing the inner surface of a mask to reduce viral and bacterial build-up after using the mask for a period of time.

Wearing a mask and social distancing are known strategies of prevention, which attempt to prevent viruses from entering the nose and the mouth. The kit and method of the invention provide an extra layer of protection against viral entry (in addition to wearing a mask, social distancing, etc.), thereby providing more prevention.

The kit and method of the invention add to those known prevention strategies by using pre-moistened swabs to topically kill viruses that may have entered the mouth and nose, before they gain a foothold in the rest of the body. Further, using pre-moistened alcohol pads to sterilize the inner surface of a mask periodically during use also helps prevent viruses and bacteria from building to dangerous levels on the inner surface of the mask.

A general aspect of the invention is a kit for providing additional defense against Corona Virus strains. The kit includes: at least one nasal swab pre-moistened with an iodine solution; and at least one oral swab pre-moistened with a chlorhexidine solution.

In some embodiments, the iodine solution of the at least one nasal swab is a betadine solution.

In some embodiments, the betadine solution is betadine 0.5-2.0%.

In some embodiments, the iodine solution of the at least one nasal swab is a povidone solution.

In some embodiments, the iodine solution of the at least one nasal swab is replaced with chlorhexidine 0.09% to 0.12%.

In some embodiments, the chlorhexidine solution of the at least one oral swab is chlorhexidine 0.09% to 0.12%.

In some embodiments, the at least one nasal swab pre-moistened with an iodine solution is sealed in a package.

In some embodiments, the package includes a plurality of nasal swabs pre-moistened with an iodine solution, and the package is resealable.

In some embodiments, a pair of nasal swabs pre-moistened with the iodine solution are included in a sealed package.

In some embodiments, the at least one oral swab pre-moistened with a chlorhexidine solution is sealed in a package.

In some embodiments, the package includes a plurality of oral swabs pre-moistened with a chlorhexidine solution, and the package is resealable.

In some embodiments, the kit also includes a plurality of alcohol swabs.

In some embodiments, the alcohol swabs are pre-moistened with 60%-70% Isopropyl Alcohol and are sealed in a disposable package.

In some embodiments, the plurality of alcohol swabs consists of four alcohol swabs.

In some embodiments, each alcohol swab includes a rectangular or square piece of gauze.

In some embodiments, each alcohol swab includes a swab stick.

In some embodiments, each alcohol swab includes a swab made from cotton.

In some embodiments, the at least one nasal swab pre-moistened with an iodine solution is sealed in a sterile package.

In some embodiments, the at least one oral swab pre-moistened with a chlorhexidine solution is sealed in a sterile package.

In some embodiments, the plurality of alcohol swabs are sealed in a sterile package.

Another general aspect of the invention is a method for providing additional defense against Corona Virus strains to a person having a mouth and a nose with two nostrils. The method includes: using at least one nasal swab pre-moistened with an iodine solution to apply the iodine solution to a surface within each of the two nostrils; and using at least one oral swab pre-moistened with a chlorhexidine solution to apply the chlorhexidine solution to a surface within the mouth.

In some embodiments, the iodine solution of the at least one nasal swab is a betadine solution.

In some embodiments, the betadine solution is betadine 0.5-2.0%.

In some embodiments, the iodine solution of the at least one nasal swab is a povidone solution.

In some embodiments, the iodine solution of the at least one nasal swab is replaced with chlorhexidine 0.09% to 0.12%.

In some embodiments, the chlorhexidine solution of the at least one oral swab is chlorhexidine 0.09% to 0.12%.

In some embodiments, the method also includes: before using the at least one nasal swab, removing the at least one nasal swab from a package.

In some embodiments, the method also includes: after removing the at least one nasal swab from the package, resealing the package.

In some embodiments, the method also includes: before using the at least one oral swab, removing the at least one oral swab from a package.

In some embodiments, the method also includes: after removing the at least one oral swab from the package, resealing the package.

### BRIEF DESCRIPTION OF THE DRAWINGS

Many additional features and advantages will become apparent to those skilled in the art upon reading the following description, when considered in conjunction with the accompanying drawings, wherein:

FIG. 1 is a side view of a person's hand holding a nasal swab pre-moistened with an iodine solution to apply the iodine solution to an inner surface of a nostril.

FIG. 2 is an enlarged side view of the nasal swab of FIG. 1 pre-moistened with an iodine solution.

FIG. 3 is a top view of two nasal swabs, each pre-moistened with an iodine solution to apply the iodine solution to an inner surface of each of two nostrils, respectively.

FIG. 4 is a top view of three pairs of nasal swabs pre-moistened with an iodine solution, each pair of pre-moistened nasal swabs sealed in a sterile package.

FIG. 5 is a top view of a pair of oral swabs pre-moistened with a chlorhexidine solution to apply the chlorhexidine solution to a surface within a mouth.

FIG. 6 is a side view of a person's hand holding an oral swab of FIG. 5 pre-moistened with a chlorhexidine solution just before applying the chlorhexidine solution to a surface within a mouth.

FIG. 7 is a side view of the person's hand of FIG. 6 holding the oral swab pre-moistened with chlorhexidine solution while applying the chlorhexidine solution to a surface within a mouth, wiping the oral swab over an inner surface of the mouth.

FIG. 8 is a front view of a person about to apply the nasal swab of FIG. 1 pre-moistened with an iodine solution to an inner surface of a nostril.

FIG. 9 is a side cross-sectional view of a person inserting the nasal swab of FIG. 1 pre-moistened with an iodine solution to apply the iodine solution to an inner surface of a nostril.

FIG. 10 is a flow chart illustrating a method for providing additional defense against Corona Virus strains using at least one nasal swab pre-moistened with an iodine solution to apply the iodine solution to an inner surface of a nostril, and using at least one oral swab pre-moistened with a chlorhexidine solution to apply the chlorhexidine solution to a surface within a mouth, returning each swab to a resealable package.

### DETAILED DESCRIPTION

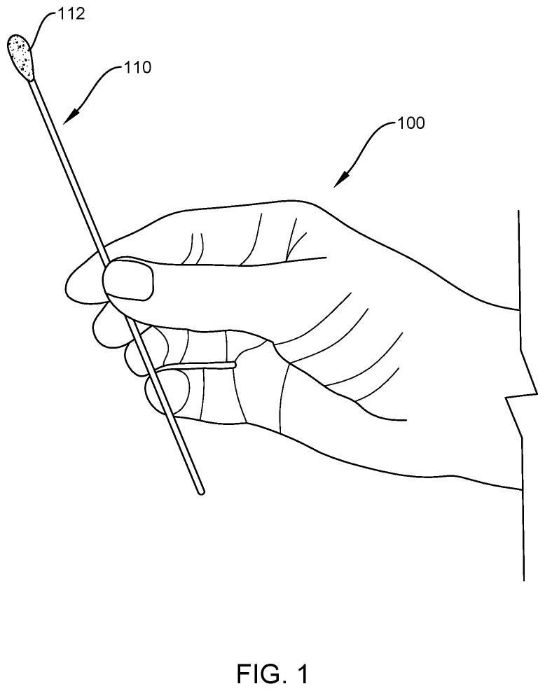

With reference to FIG. 1, a person's hand 100 is shown holding a nasal swab 110 pre-moistened with an iodine solution 112 to apply the iodine solution 112 to an inner surface of a nostril.

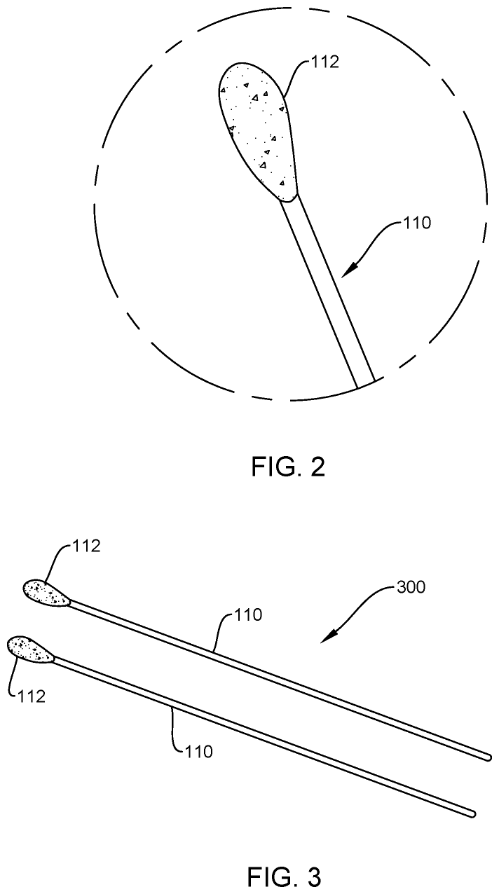

Referring to FIG. 2, the nasal swab 110 of FIG. 1 is shown, pre-moistened with an iodine solution 112.

With reference to FIG. 3, a pair 300 of two nasal swabs 110 are shown, each pre-moistened with an iodine solution 112 to apply the iodine solution 112 to an inner surface of each of two nostrils, respectively.

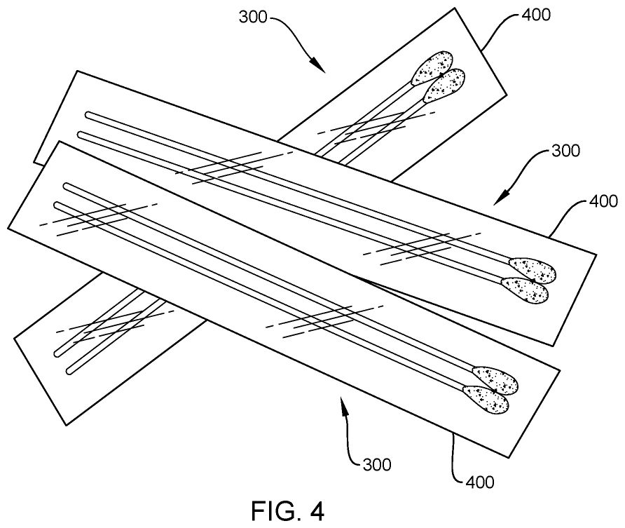

Referring to FIG. 4, three pairs 300 of nasal swabs are shown pre-moistened with an iodine solution, each pair 300 of pre-moistened nasal swabs is sealed in a sterile package 400.

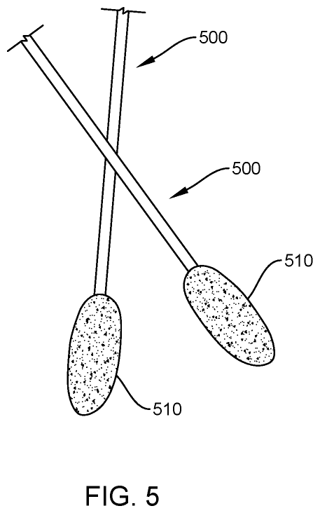

With reference to FIG. 5, a pair of oral swabs 500 is shown pre-moistened with a chlorhexidine solution 510 to apply the chlorhexidine solution 510 to a surface within a mouth.

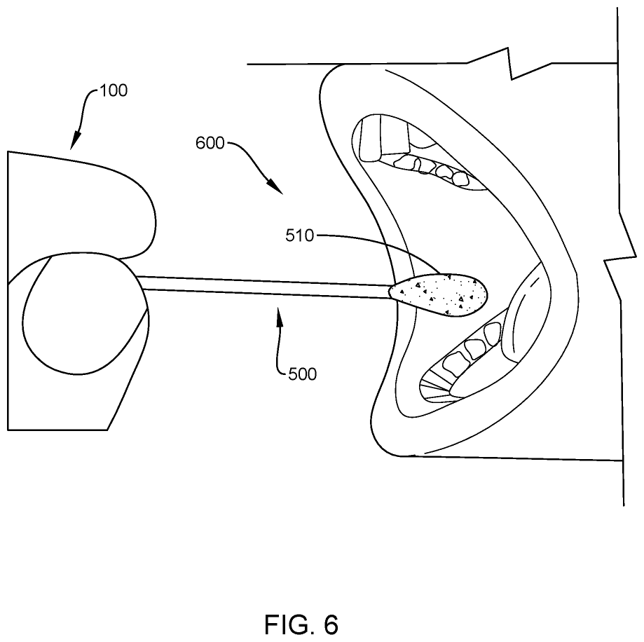

Referring to FIG. 6, a person's hand 100 is shown holding an oral swab 500 of FIG. 5 pre-moistened with a chlorhexidine solution 510 just before applying the chlorhexidine solution to a surface within a mouth 600.

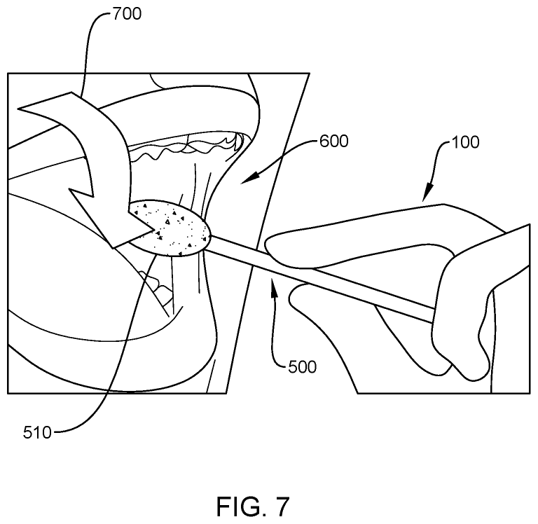

With reference to FIG. 7, the person's hand 100 of FIG. 6 is shown holding the oral swab 500 pre-moistened with chlorhexidine solution 510 while applying the chlorhexidine solution 510 to a surface within a mouth 600, using a wiping motion 700 of the oral swab 500 over an inner surface of the mouth 600.

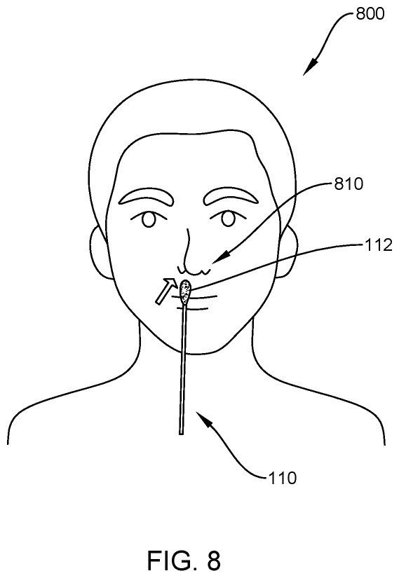

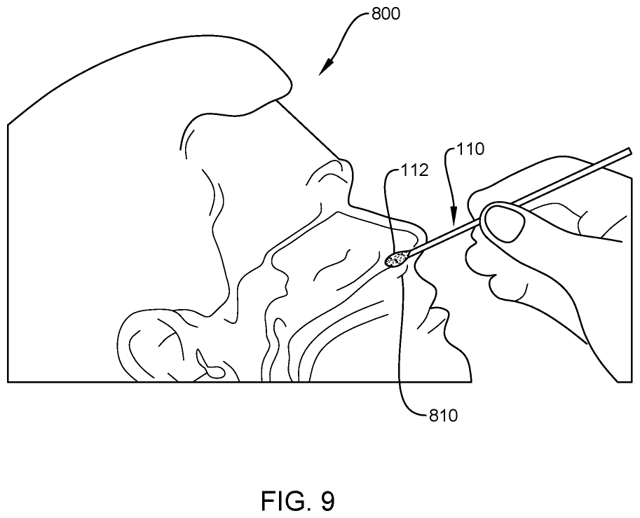

Referring to FIG. 8, a person 800 is shown about to insert the nasal swab 110 of FIG. 1 so as to apply the iodine solution 112 to an inner surface of a nostril 810.

With reference to FIG. 9, the person 800 is shown inserting the nasal swab 110 of FIG. 1 pre-moistened with an iodine solution 112 to apply the iodine solution 112 to the inner surface of the nostril 810.

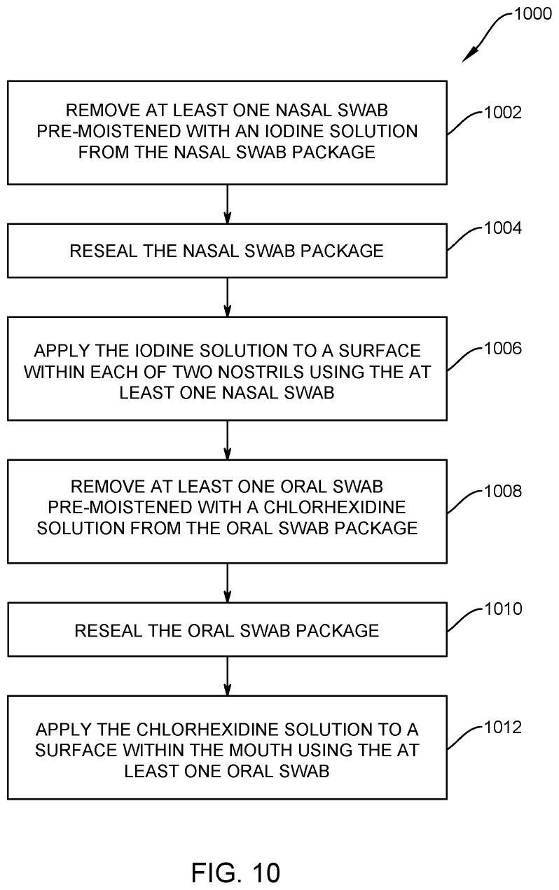

Referring to FIG. 10, a flow chart of a method 1000 is shown for providing additional defense against Corona Virus strains. First, at least one nasal swab pre-moistened with an iodine solution is removed 1002 from a package containing nasal swabs. Next, the package containing nasal swabs is resealed 1004 to keep the remaining nasal swabs fresh and sterile. Then, the iodine solution is applied 1006 to an inner surface of a nostril. Further, an oral swab pre-moistened with a chlorhexidine solution is removed 1008 from a package containing oral swabs. Next, the package containing oral swabs is resealed 1010 to keep the remaining oral swabs fresh and sterile. Then, the chlorhexidine solution is applied 1012 to a surface within a mouth using the oral swab, returning each swab to a resealable package.

The kit and method of the invention include the use of 4″ long cotton nasal swabs pre-moistened with betadine or povidone iodine solution. A person inserts a nasal swab into each nostril, using one nasal swab per nostril. The nasal swabs are sterile and are pre-moistened with 2% betadine, for example, or povidone iodine solution.

An oral swab that is 6″ long is also included that is pre-moistened in 0.12% chlorhexidine solution, for example. The chlorhexidine can be 0.09% to 0.12%. Usually, 0.12% chlorhexidine solution can be used.

The povidone iodine concentration can be 0.5%, 1.0%, or 2.0%, depending on the level of prevention desired. Usually, 0.5% povidone iodine solution can be used.

If one wants to create a pre-moistened nasal swab, a dry cotton swab can be dipped in 2 ml of 0.5% povidone iodine solution for 15 seconds. If one wants to create a pre-moistened oral swab, a dry cotton swab can be dipped in 15 ml of 0.12% chlorhexidine solution for 15 seconds.

Each of the two nasal swabs is to be pre-moistened in 2 mL of betadine solution, so 4 mL total of betadine solution would be used to pre-moisten two nasal swabs. The minimum number of nasal swabs in a kit is two, and the minimum number or oral swabs in a kit is one.

Optionally, for further protection, alcohol swabs can be used for sterilizing the inner surface of a mask. Four alcohol pads (pre-moistened with 70% alcohol) can optionally be part of the kit.

Other modifications and implementations will occur to those skilled in the art without departing from the spirit and the scope of the invention as claimed. Accordingly, the above description is not intended to limit the invention, except as indicated in the following claims.

1. A kit for providing additional defense against Corona Virus strains, the kit comprising:
at least one nasal swab pre-moistened with an iodine solution and coupled to a handle; and
at least one oral swab pre-moistened with a chlorhexidine solution and coupled to a handle.

2. The kit of claim 1, wherein the iodine solution of the at least one nasal swab is a betadine solution.

3. The kit of claim 2, wherein the betadine solution is betadine 0.5-2.0%.

4. The kit of claim 1, wherein the iodine solution of the at least one nasal swab is a povidone solution.

5. The kit of claim 1, wherein the iodine solution of the at least one nasal swab is replaced with chlorhexidine 0.09% to 0.12%.

6. The kit of claim 1, wherein the chlorhexidine solution of the at least one oral swab is chlorhexidine 0.09% to 0.12%.

7. The kit of claim 1, wherein the at least one nasal swab pre-moistened with an iodine solution is sealed in a package.

8. The kit of claim 7, wherein the package includes a plurality of nasal swabs pre-moistened with an iodine solution and each coupled to a handle, and the package is resealable.

9. The kit of claim 1, wherein a pair of nasal swabs pre-moistened with the iodine solution and coupled to a handle are included in a sealed package.

10. The kit of claim 1, wherein the at least one oral swab pre-moistened with a chlorhexidine solution and coupled to a handle is sealed in a package.

11. The kit of claim 10, wherein the package includes a plurality of oral swabs pre-moistened with a chlorhexidine solution and coupled to a handle, and the package is resealable.

12. The kit of claim 1, further comprising: a plurality of alcohol swabs.

13. The kit of claim 12, wherein the alcohol swabs are pre-moistened with 60%-70% Isopropyl Alcohol and are sealed in a disposable package.

14. The kit of claim 12, wherein the plurality of alcohol swabs consists of four alcohol swabs.

15. The kit of claim 12, wherein each alcohol swab comprises a rectangular or square piece of gauze.

16. The kit of claim 12, wherein each alcohol swab is coupled to a handle.

17. The kit of claim 12, wherein each alcohol swab includes a swab made from cotton.

18. The kit of claim 7, wherein the at least one nasal swab pre-moistened with an iodine solution is sealed in a sterile package.

19. The kit of claim 10, wherein the at least one oral swab pre-moistened with a chlorhexidine solution is sealed in a sterile package.

20. The kit of claim 12, wherein the plurality of alcohol swabs is sealed in a sterile package.

21. A method for providing additional defense against Corona Virus strains to a person having a mouth and a nose with two nostrils, the method comprising:
using at least one nasal swab pre-moistened with an iodine solution to apply the iodine solution to a surface within each of the two nostrils; and
using at least one oral swab pre-moistened with a chlorhexidine solution to apply the chlorhexidine solution to a surface within the mouth.

22. The method of claim 21, wherein the iodine solution of the at least one nasal swab is a betadine solution.

23. The method of claim 22, wherein the betadine solution is betadine 0.5-2.0%.

24. The method of claim 21, wherein the iodine solution of the at least one nasal swab is a povidone solution.

25. The method of claim 21, wherein the iodine solution of the at least one nasal swab is replaced with chlorhexidine 0.09% to 0.12%.

26. The method of claim 21, wherein the chlorhexidine solution of the at least one oral swab is chlorhexidine 0.09% to 0.12%.

27. The method of claim 21, further comprising: before using the at least one nasal swab, removing the at least one nasal swab from a package.

28. The method of claim 27, further comprising: after removing the at least one nasal swab from the package, resealing the package.

29. The method of claim 21, further comprising: before using the at least one oral swab, removing the at least one oral swab from a package.

30. The method of claim 29, further comprising: after removing the at least one oral swab from the package, resealing the package.
